(19) United States
(12) Patent Application Publication
(10) Pub. No.: US 2025/0029257 A1
(43) Pub. Date: Jan. 23, 2025

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventor: Yu HASEGAWA, Tokyo (JP)

(21) Appl. No.: 18/905,154

(22) Filed: Oct. 3, 2024

Related U.S. Application Data
(63) Continuation of application No. PCT/JP2023/014935, filed on Apr. 12, 2023.

(30) Foreign Application Priority Data
Apr. 12, 2022 (JP) 2022-065907

(51) Int. Cl. G06T 7/00; G06V 10/25

(52) U.S. Cl. CPC G06T 7/0014; G06V 10/25; G06T 2207/30096

(57) ABSTRACT

An information processing apparatus including a processor, wherein the processor is configured to: acquire a character string including a description regarding at least one first image obtained by imaging a subject at a first point in time; specify a first region of interest described in the character string; specify a first image of interest including the first region of interest from the first image; specify a second image of interest corresponding to the first image of interest from at least one second image obtained by imaging the subject at a second point in time; and display the first image of interest and the second image of interest on a display in association with each other.

### CROSS REFERENCE TO RELATED APPLICATIONS

This application is a continuation of International Application No. PCT/JP2023/014935, filed on Apr. 12, 2023, which claims priority from Japanese Patent Application No. 2022-065907, filed on Apr. 12, 2022. The entire disclosure of each of the above applications is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. Further, medical images are analyzed via computer-aided detection/diagnosis (CAD) using a discriminator in which learning is performed by deep learning or the like, and regions of interest including structures, lesions, and the like included in the medical images are detected and/or diagnosed. The medical images and analysis results via CAD are transmitted to a terminal of a healthcare professional such as a radiologist who interprets the medical images. The healthcare professional such as a radiologist interprets the medical images by referring to the medical images and analysis results using his or her own terminal and creates an interpretation report.

In addition, various methods have been proposed to support the creation of interpretation reports in order to reduce the burden of the interpretation work. For example, JP2019-153250A discloses a technology for creating an interpretation report based on a keyword input by a radiologist and on an analysis result of a medical image. In the technology disclosed in JP2019-153250A, a sentence to be included in the interpretation report is created by using a recurrent neural network trained to generate a sentence from input characters.

Further, for example, in regular health checkups and post-treatment follow-up observations, the same subject may be examined a plurality of times, and a change over time in a medical condition may be checked by performing comparative interpretation of medical images at each point in time. Therefore, various methods for performing comparative interpretation have been proposed. For example, JP2005-012248A discloses a method for performing registration on images by calculating an index value representing a degree of matching between a plurality of past images and a plurality of current images for all combinations of the two groups of images and extracting the combination with the highest degree of matching.

In the case of creating an interpretation report at a current point in time by performing comparative interpretation of medical images at a past point in time and the current point in time, not only the medical images but also the interpretation reports created at the past point in time may be referred to. Therefore, there is a demand for a technology that enables comparative interpretation of medical images at the past point in time and the current point in time for a region of interest described in an interpretation report at the past point in time.

### SUMMARY

The present disclosure provides an information processing apparatus, an information processing method, and an information processing program that can support creation of an interpretation report.

According to a first aspect of the present disclosure, there is provided an information processing apparatus comprising at least one processor, in which the processor is configured to: acquire a character string including a description regarding at least one first image obtained by imaging a subject at a first point in time; specify a first region of interest described in the character string; specify a first image of interest including the first region of interest from the first image; specify a second image of interest corresponding to the first image of interest from at least one second image obtained by imaging the subject at a second point in time; and display the first image of interest and the second image of interest on a display in association with each other.

According to a second aspect of the present disclosure, in the first aspect, the processor may be configured to specify the second image obtained by imaging the same position as the first image of interest as the second image of interest.

According to a third aspect of the present disclosure, in the first or second aspect, the processor may be configured to receive a selection of a portion of the character string to be used to specify the first region of interest.

According to a fourth aspect of the present disclosure, in any one of the first to third aspects, the processor may be configured to, in a case in which a plurality of the first regions of interest described in the character string are specified, specify the first image of interest and the second image of interest for each of the plurality of first regions of interest.

According to a fifth aspect of the present disclosure, in the fourth aspect, the processor may be configured to display the first image of interest and the second image of interest specified for each of the plurality of first regions of interest on the display in sequence.

According to a sixth aspect of the present disclosure, in the fifth aspect, the processor may be configured to display the first image of interest and the second image of interest on the display in sequence in an order according to a predetermined priority for each of the plurality of first regions of interest.

According to a seventh aspect of the present disclosure, in the fifth or sixth aspect, the processor may be configured to: display an input field for receiving a character string including a description regarding the second image of interest on the display in association with the second image of interest; and display a next first image of interest and a next second image of interest on the display after receiving the character string including the description regarding the second image of interest in the input field.

According to an eighth aspect of the present disclosure, in the fourth aspect, the processor may be configured to display the first image of interest and the second image of interest specified for each of the plurality of first regions of interest on the display as a list. According to a ninth aspect of the present disclosure, in any one of the first to eighth aspects, the processor may be configured to notify a user to check a region corresponding to the first region of interest in the second image of interest.

According to a tenth aspect of the present disclosure, in the ninth aspect, the processor may be configured to display a character string indicating the first region of interest and at least one of a symbol or a figure on the display as the notification.

According to an eleventh aspect of the present disclosure, in any one of the first to tenth aspects, the processor may be configured to: generate comparison information indicating a result of comparing the first region of interest in the first image of interest with a region corresponding to the first region of interest in the second image of interest; and display the comparison information on the display.

According to a twelfth aspect of the present disclosure, in any one of the first to eleventh aspects, the processor may be configured to highlight a region corresponding to the first region of interest in the second image of interest.

According to a thirteenth aspect of the present disclosure, in any one of the first to twelfth aspects, the processor may be configured to display a character string including a description regarding at least the first region of interest on the display in association with the first image of interest.

According to a fourteenth aspect of the present disclosure, in any one of the first to thirteenth aspects, the processor may be configured to display an input field for receiving a character string including a description regarding the second image of interest on the display in association with the second image of interest.

According to a fifteenth aspect of the present disclosure, in any one of the first to fourteenth aspects, the processor may be configured to display the first image of interest and the second image of interest on the display with the same display settings.

According to a sixteenth aspect of the present disclosure, in the fifteenth aspect, the display settings may be settings related to at least one of a resolution, a gradation, a brightness, a contrast, a window level, a window width, or a color of the first image of interest and the second image of interest.

According to a seventeenth aspect of the present disclosure, in any one of the first to sixteenth aspects, the processor may be configured to, in a case in which a region corresponding to the first region of interest is not included in the second image of interest, provide a notification indicating that the region corresponding to the first region of interest is not included in the second image of interest.

According to an eighteenth aspect of the present disclosure, in any one of the first to seventeenth aspects, the first image and the second image may be medical images, and the first region of interest may be at least one of a region of a structure included in the medical image or a region of an abnormal shadow included in the medical image.

According to a nineteenth aspect of the present disclosure, there is provided an information processing method comprising: acquiring a character string including a description regarding at least one first image obtained by imaging a subject at a first point in time; specifying a first region of interest described in the character string; specifying a first image of interest including the first region of interest from the first image; specifying a second image of interest corresponding to the first image of interest from at least one second image obtained by imaging the subject at a second point in time; and displaying the first image of interest and the second image of interest on a display in association with each other.

According to a twentieth aspect of the present disclosure, there is provided an information processing program for causing a computer to execute a process comprising: acquiring a character string including a description regarding at least one first image obtained by imaging a subject at a first point in time; specifying a first region of interest described in the character string; specifying a first image of interest including the first region of interest from the first image; specifying a second image of interest corresponding to the first image of interest from at least one second image obtained by imaging the subject at a second point in time; and displaying the first image of interest and the second image of interest on a display in association with each other.

The information processing apparatus, the information processing method, and the information processing program according to the aspects of the present disclosure can support creation of an interpretation report.

### BRIEF DESCRIPTION OF THE DRAWINGS

FIG. 1 is a diagram showing an example of a schematic configuration of an information processing system.

FIG. 2 is a diagram showing an example of a medical image.

FIG. 3 is a diagram showing an example of a medical image.

FIG. 4 is a block diagram showing an example of a hardware configuration of an information processing apparatus.

FIG. 5 is a block diagram showing an example of a functional configuration of the information processing apparatus.

FIG. 6 is a diagram showing an example of a comment on findings.

FIG. 7 is a diagram showing an example of a screen displayed on a display.

FIG. 8 is a diagram showing an example of a screen displayed on a display.

FIG. 9 is a flowchart showing an example of information processing.

FIG. 10 is a diagram showing an example of a screen displayed on a display.

FIG. 11 is a diagram showing an example of a screen displayed on a display.

FIG. 12 is a diagram showing an example of a screen displayed on a display.

FIG. 13 is a diagram showing an example of a screen displayed on a display.

### DETAILED DESCRIPTION

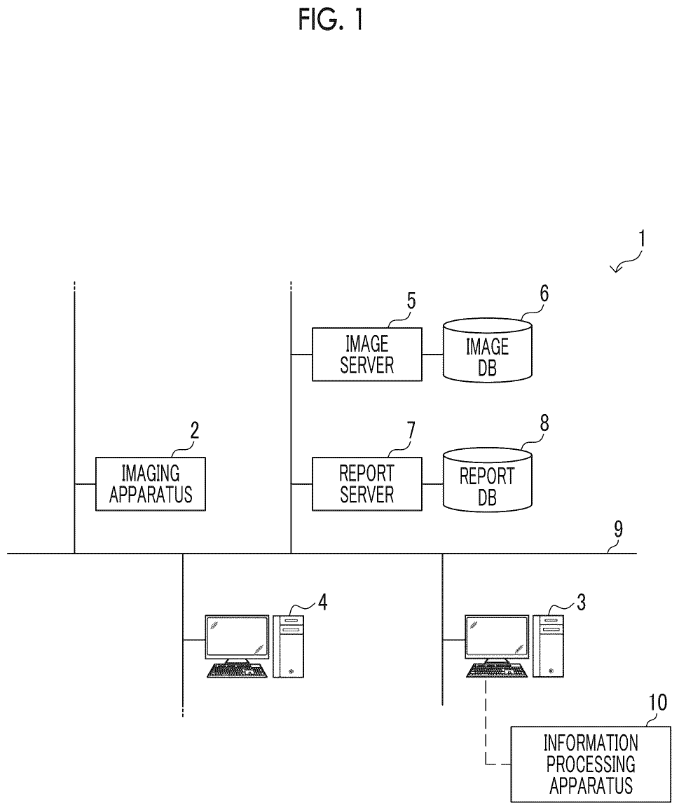

Hereinafter, embodiments of the present disclosure will be described with reference to the drawings. First, a configuration of an information processing system 1 to which an information processing apparatus of the present disclosure is applied will be described. FIG. 1 is a diagram showing a schematic configuration of the information processing system 1. The information processing system 1 shown in FIG. 1 performs imaging of an examination target part of a subject and storing of a medical image acquired by the imaging based on an examination order from a doctor in a medical department using a known ordering system. In addition, the information processing system 1 performs interpretation work of a medical image and creation of an interpretation report by a radiologist and viewing of the interpretation report by a doctor of a medical department that is a request source.

As shown in FIG. 1, the information processing system 1 includes an imaging apparatus 2, an interpretation workstation (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc read-only memory (DVD-ROM) or a compact disc read-only memory (CD-ROM), and distributed, and be installed on the computer from the recording medium. In addition, the application program may be stored in, for example, a storage device of a server computer connected to the network 9 or in a network storage in a state in which it can be accessed from the outside, and be downloaded and installed on the computer in response to a request.

The imaging apparatus 2 is an apparatus (modality) that generates a medical image T showing a diagnosis target part of the subject by imaging the diagnosis target part. Examples of the imaging apparatus 2 include a simple X-ray imaging apparatus, a computed tomography (CT) apparatus, a magnetic resonance imaging (MRI) apparatus, a positron emission tomography (PET) apparatus, an ultrasound diagnostic apparatus, an endoscope, a fundus camera, and the like. The medical image generated by the imaging apparatus 2 is transmitted to the image server 5 and is stored in the image DB 6.

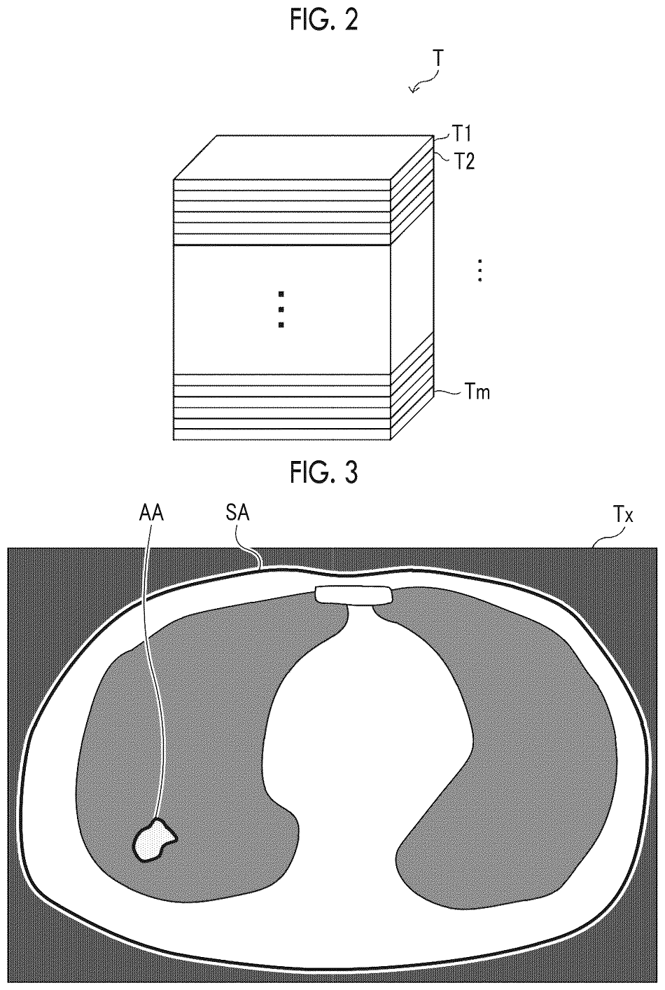

FIG. 2 is a diagram schematically showing an example of a medical image acquired by the imaging apparatus 2. A medical image T shown in FIG. 2 is, for example, a CT image consisting of a plurality of tomographic images T1 to Tm (m is 2 or more) representing tomographic planes from a head to a waist of one subject (human body).

FIG. 3 is a diagram schematically showing an example of one tomographic image Tx out of the plurality of tomographic images T1 to Tm. The tomographic image Tx shown in FIG. 3 represents a tomographic plane including lungs. Each of the tomographic images Tl to Tm may include a region SA of a structure showing various organs and viscera of the human body (for example, lungs, livers, and the like), various tissues constituting various organs and viscera (for example, blood vessels, nerves, muscles, and the like), and the like. In addition, each tomographic image may include a region AA of an abnormal shadow showing lesions such as, for example, nodules, tumors, injuries, defects, and inflammation. In the tomographic image Tx shown in FIG. 3, a lung region is the region SA of the structure, and a nodule region is the region AA of the abnormal shadow. A single tomographic image may include regions SA of a plurality of structures and/or regions AA of a plurality of abnormal shadows. Hereinafter, at least one of the region SA of the structure included in the medical image or the region AA of the abnormal shadow included in the medical image will be referred to as a “region of interest”.

The interpretation WS 3 is a computer used by, for example, a healthcare professional such as a radiologist of a radiology department to interpret a medical image and to create an interpretation report, and encompasses an information processing apparatus 10 according to the present embodiment. In the interpretation WS 3, a viewing request for a medical image to the image server 5, various types of image processing for the medical image received from the image server 5, display of the medical image, and input reception of a sentence regarding the medical image are performed. In the interpretation WS 3, analysis processing for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing device, a display device such as a display, and an input device such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network-attached storage (NAS) and a storage area network (SAN).

The image DB 6 is realized by, for example, a storage medium such as a hard disk drive (HDD), a solid-state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as an imaging method, an imaging condition, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, a slice thickness in tomographic imaging, and the like. In addition, the accessory information may include information related to the subject such as the name, date of birth, age, and gender of the subject. In addition, the accessory information may include information regarding the imaging purpose of the medical image.

In a case in which the image server 5 receives a request to register a medical image from the imaging apparatus 2, the image server 5 prepares the medical image in a format for a database and registers the medical image in the image DB 6. In addition, in a case in which the viewing request from the interpretation WS 3 and the medical care WS 4 is received, the image server 5 searches for a medical image registered in the image DB 6 and transmits the found medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered. In addition, the report DB 8 may store finding information regarding the medical image. Finding information includes, for example, information obtained by the interpretation WS 3 through image analysis of a medical image using a computer-aided detection/diagnosis (CAD) technology, an artificial intelligence (AI) technology, or the like, and information or the like input by a user after interpreting a medical image.

For example, finding information includes information indicating various findings such as a name (type), a property, a position, a measurement value, and an estimated disease name of a region of interest included in the medical image. Examples of names (types) include the names of structures such as “lung” and “liver”, and the names of abnormal shadows such as “nodule”. The property mainly means the features of abnormal shadows. For example, in the case of a lung nodule, findings indicating opacity such as “solid” and “ground-glass”, margin shapes such as “well-defined/ill-defined”, “smooth/irregular”, “spicula”, “lobulated”, and “lagged”, and an overall shape such as “round” and “irregular form” can be mentioned. Also, for example, the relationship with the peripheral tissue, such as “pleural contact” and “pleural invagination”, and findings regarding the presence or absence of contrast, washout, and the like can be mentioned.

The position means an anatomical position, a position in a medical image, and a relative positional relationship with other regions of interest such as “inside”, “margin”, and “periphery”. The anatomical position may be indicated by an organ name such as “lung” and “liver”, and may be expressed in terms of subdivided lungs such as “right lung”, “upper lobe”, and apical segment (“S1”). The measurement value is a value that can be quantitatively measured from a medical image, and is, for example, at least one of a size or a signal value of a region of interest. The size is represented by, for example, a major axis, a minor axis, an area, a volume, or the like of a region of interest. The signal value is represented by, for example, a pixel value in a region of interest, a CT value in units of HU, or the like. The estimated disease name is an evaluation result estimated based on the abnormal shadow. Examples of the estimated disease name include a disease name such as “cancer” and “inflammation” and an evaluation result such as “negative/positive”, “benign/malignant”, and “mild/severe” related to disease names and properties.

Further, in a case in which the report server 7 receives a request to register the interpretation report from the interpretation WS 3, the report server 7 prepares the interpretation report in a format for a database and registers the interpretation report in the report DB 8. Further, in a case in which the report server 7 receives the viewing request for the interpretation report from the interpretation WS 3 and the medical care WS 4, the report server 7 searches for the interpretation report registered in the report DB 8, and transmits the found interpretation report to the interpretation WS 3 and to the medical care WS 4 that are viewing request sources.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

For example, in regular health checkups and post-treatment follow-up observations, the same subject may be examined a plurality of times and a change over time in a medical condition may be checked by performing comparative interpretation of medical images at each point in time. In addition, in the case of creating an interpretation report at a current point in time, not only medical images but also interpretation reports created at a past point in time may be referred to. Therefore, the information processing apparatus 10 according to the present embodiment has a function of enabling comparative interpretation of a medical image at a past point in time and a medical image at a current point in time for a region of interest described in an interpretation report at the past point in time. The information processing apparatus 10 will be described below. As described above, the information processing apparatus 10 is encompassed in the interpretation WS 3.

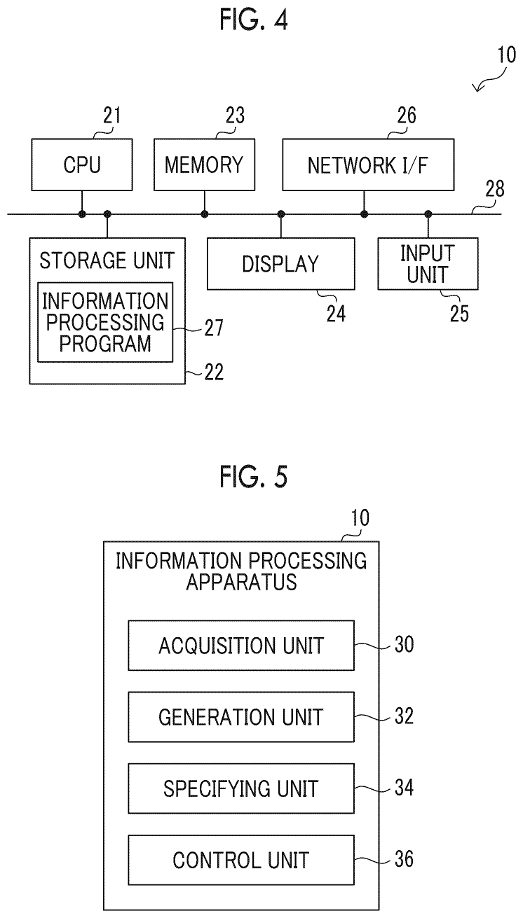

First, with reference to FIG. 4, an example of a hardware configuration of the information processing apparatus 10 according to the present embodiment will be described. As shown in FIG. 4, the information processing apparatus 10 includes a central processing unit (CPU) 21, a non-volatile storage unit 22, and a memory 23 as a temporary storage area. Further, the information processing apparatus 10 includes a display 24 such as a liquid-crystal display, an input unit 25 such as a keyboard and a mouse, and a network interface (I/F) 26. The network I/F 26 is connected to the network 9 and performs wired or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. As the information processing apparatus 10, for example, a personal computer, a server computer, a smartphone, a tablet terminal, a wearable terminal, or the like can be applied as appropriate.

Next, with reference to FIGS. 5 to 8, an example of a functional configuration of the information processing apparatus 10 according to the present embodiment will be described. As shown in FIG. 5, the information processing apparatus 10 includes an acquisition unit 30, a generation unit 32, a specifying unit 34, and a control unit 36. In a case in which the CPU 21 executes the information processing program 27, the CPU 21 functions as each of the functional units of the acquisition unit 30, the generation unit 32, the specifying unit 34, and the control unit 36.

The acquisition unit 30 acquires at least one medical image (hereinafter referred to as a “first image”) obtained by imaging a subject at a past point in time from the image server 5. The acquisition unit 30 also acquires at least one medical image (hereinafter referred to as a “second image”) obtained by imaging the subject at a current point in time from the image server 5. The subject to be imaged in the first image and the second image is the same subject. Hereinafter, an example in which the acquisition unit 30 acquires a plurality of tomographic images included in a CT image captured at a past point in time as a plurality of first images, and acquires a plurality of tomographic images included in a CT image captured at a current point in time as a plurality of second images will be described (see FIG. 2). The past point in time is an example of a first point in time of the present disclosure, and the current point in time is an example of a second point in time of the present disclosure.

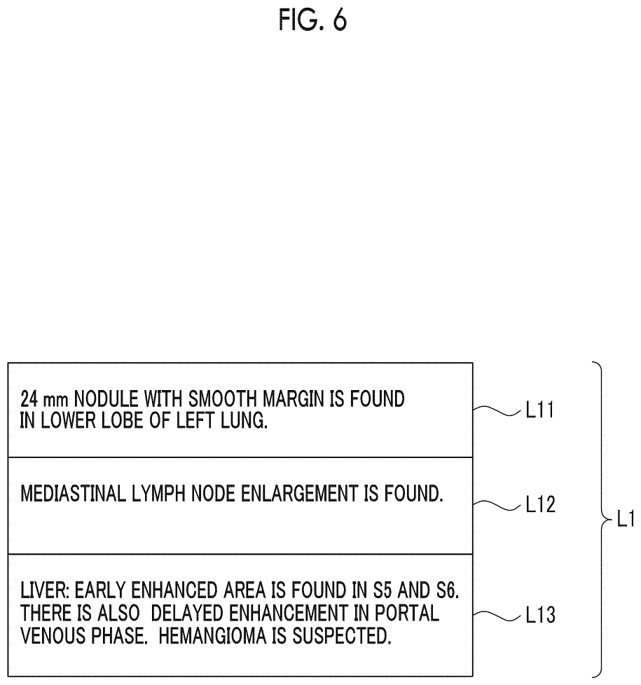

Furthermore, the acquisition unit 30 acquires a character string including a description regarding the first image, which was created at a past point in time, from the report server 7. FIG. 6 shows a comment on findings L1 as an example of a character string. The comment on findings L1 includes a plurality of comments on findings, namely, a comment on findings L11 regarding a nodule in a lung field, a comment on findings L12 regarding a mediastinal lymph node enlargement, and a comment on findings L13 regarding a hemangioma in the liver. In this manner, the character string acquired by the acquisition unit 30 may include descriptions of a plurality of regions of interest (for example, lesions and structures). In addition, a character string may be, for example, a document such as an interpretation report, a sentence such as a comment on findings included in a document such as an interpretation report, text including a plurality of sentences, and words included in a sentence, text, or a document. Also, for example, the character string may be a character string indicating the finding information stored in the report DB 8.

The specifying unit 34 specifies a first region of interest described in the character string such as a comment on findings acquired by the acquisition unit 30. Furthermore, the specifying unit 34 may specify a plurality of first regions of interest described in a character string such as a comment on findings. For example, the specifying unit 34 may extract words representing the names (types) of lesions and structures, such as “lower lobe of left lung”, “nodule”, “mediastinal lymph node enlargement”, “liver”, and “hemangioma” from the comment on findings L1 to specify these as the first region of interest. As a method for extracting words from character strings such as comments on findings, a known named entity extraction method using a natural language processing model, such as, for example, bidirectional encoder representations from transformers (BERT), can be applied as appropriate.

Furthermore, the specifying unit 34 specifies a first image of interest including the first region of interest specified from the character string, such as the comments on findings, from the first image acquired by the acquisition unit 30. For example, the specifying unit 34 may extract a region of interest included in each of a plurality of first images (tomographic images) by performing image analysis on each of the first images, and specify a first image including a region of interest that substantially matches the first region of interest specified from a character string such as a comment on findings, as the first image of interest. For example, the specifying unit 34 may specify a first image showing a tomographic plane including the “nodule” of the “lower lobe of left lung” specified from the comment on findings L11 as a first image of interest T11.

As a method for extracting a region of interest included in the first image, a known method using a CAD technology, an AI technology, or the like can be appropriately applied. For example, the specifying unit 34 may extract a region of interest included in the first image by using a learning model such as a convolutional neural network (CNN) that has been trained to receive the medical image as an input and extract and output a region of interest included in the medical image. Note that, by extracting the region of interest included in the first image, the position of the first region of interest in the first image of interest is also specified.

In addition, the specifying unit 34 specifies a second image of interest corresponding to the first image of interest from the second image acquired by the acquisition unit 30. Specifically, the specifying unit 34 specifies the second image obtained by imaging the same position as the specified first image of interest as the second image of interest. As a method for specifying the second image obtained by imaging the same position as the first image of interest, for example, a known registration method such as the technology disclosed in JP2005-012248A or the like can be applied as appropriate.

Furthermore, in a case in which the specifying unit 34 specifies a plurality of first regions of interest from a character string such as a comment on findings, the specifying unit 34 may specify a first image of interest and a second image of interest for each of the specified plurality of first regions of interest. This is because the first image and the second image included in each of the plurality of first regions of interest may be different from one another. For example, in addition to the first image of interest T11 including the “nodule” of the “lower lobe of left lung”, the specifying unit 34 may specify a first image showing a tomographic plane including the “mediastinal lymph node enlargement” specified from the comment on findings L12 as another first image of interest T12. Furthermore, the specifying unit 34 may specify a first image showing a tomographic plane including the “liver” and the “hemangioma” specified from the comment on findings L13 as another first image of interest T13.

The generation unit 32 generates a character string such as a comment on findings regarding the second image of interest specified by the specifying unit 34. Specifically, first, the generation unit 32 extracts a region corresponding to the first region of interest in the second image of interest (hereinafter referred to as a “second region of interest”). For example, the generation unit 32 may extract a second region of interest included in the second image of interest by using a learning model such as a CNN that has been trained to receive the medical image as an input and extract and output a region of interest included in the medical image. Also, for example, a region in the second image of interest at the same position as the first region of interest in the first image of interest specified by the specifying unit 34 may be extracted as the second region of interest.

Thereafter, the generation unit 32 performs image analysis on the extracted second region of interest to generate finding information of the second region of interest. As a method for acquiring finding information via image analysis, a known method using a CAD technology, an AI technology, or the like can be appropriately applied. For example, the generation unit 32 may generate finding information of a second region of interest by using a learning model such as a CNN that has been trained in advance to receive the region of interest extracted from the medical image as an input and output the finding information of the region of interest.

Thereafter, the generation unit 32 generates a character string such as a comment on findings including the generated finding information of the second region of interest. For example, the generation unit 32 may generate a comment on findings by using a method using machine learning such as the recurrent neural network described in JP2019-153250A. Further, for example, the generation unit 32 may generate a comment on findings by embedding finding information in a predetermined template. Further, for example, the generation unit 32 may generate a comment on findings for the second region of interest by reusing a character string such as a comment on findings including a description regarding the first image acquired by the acquisition unit 30 and correcting a portion corresponding to the changed finding information.

Furthermore, the generation unit 32 may generate comparison information indicating a result of comparing the first region of interest in the first image of interest with the second region of interest in the second image of interest. For example, the generation unit 32 may generate comparison information indicating variations in measurement values such as the size and signal values of each region of interest, as well as changes over time such as improvement or deterioration of properties, based on finding information of the first region of interest and the second region of interest. For example, in a case in which the size of the second region of interest is larger than the size of the first region of interest, the generation unit 32 may generate comparison information indicating that the sizes are tending to increase. The generation unit 32 may generate a character string such as a comment on findings including comparison information, or may generate a graph showing variations in measurement values such as the size and signal values.

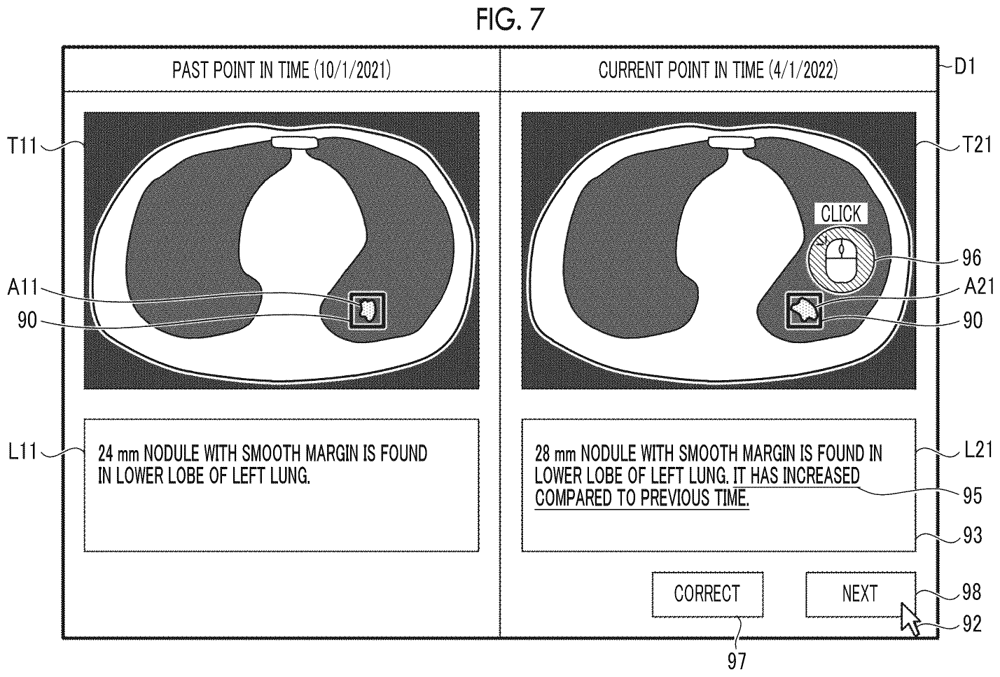

The control unit 36 performs control to display the first image of interest and the second image of interest specified by the specifying unit 34 on the display 24 in association with each other. FIG. 7 shows an example of a screen D1 displayed on the display 24 by the control unit 36. On the screen D1, a first image of interest T11 including a nodule A11 in the lower lobe of the left lung (an example of a first region of interest) specified from the comment on findings L11 of FIG. 6, and a second image of interest T21 corresponding to the first image of interest T11 are displayed. As shown on the screen D1, the control unit 36 may facilitate comparative interpretation by displaying the first image of interest T11 and the second image of interest T21 specified by the specifying unit 34 side by side.

Furthermore, the control unit 36 may highlight at least one of the first region of interest in the first image of interest or the second region of interest in the second image of interest. For example, as shown on the screen D1, the control unit 36 may surround the nodule A11 (first region of interest) in the first image of interest T11 and a nodule A21 (second region of interest) in the second image of interest T21 with respective bounding boxes 90. For example, the control unit 36 may also add a marker such as an arrow near the first region of interest and/or the second region of interest, color-code the first region of interest and/or the second region of interest from other regions, or enlarge and display the first region of interest and/or the second region of interest.

Furthermore, the control unit 36 may notify the user to check the second region of interest in the second image of interest. For example, the control unit 36 may display at least one of a character string, a symbol, or a figure indicating the first region of interest near the nodule A21 (second region of interest) in the second image of interest T21 on the display 24 as the notification. For example, on the screen D1, an icon 96 is shown near the nodule A21. In addition, for example, the control unit 36 may provide a notification through sound output from a speaker and by means such as blinking of a light source like a light bulb or a light-emitting diode (LED).

Furthermore, the control unit 36 may perform control to display the first image of interest and the second image of interest on the display 24 with the same display settings. The display settings are, for example, settings related to at least one of a resolution, a gradation, a brightness, a contrast, a window level (WL), a window width (WW), or a color of the first image of interest and the second image of interest. The window level is a parameter related to the gradation of a CT image, and is the central value of the CT values displayed on the display 24. The window width is a parameter related to the gradation of a CT image, and is the width between the lower limit value and the upper limit value of the CT value displayed on the display 24. For example, even for CT images of the same position, the display settings suitable for observing the lung field and the display settings suitable for observing the mediastinum are different. It is preferable that the control unit 36 sets the display settings of the first image of interest and the second image of interest, which are to be displayed in association with each other on the display 24, to be the same, thereby facilitating comparative interpretation.

The control unit 36 may also perform control to display a character string such as a comment on findings including a description regarding at least the first region of interest acquired by the acquisition unit 30 on the display 24 in association with the first image of interest. On the screen D1, the comment on findings L11 regarding the nodule A11 (first region of interest) is displayed below the first image of interest T11.

The control unit 36 may also perform control to display a character string such as a comment on findings including the finding information of the second region of interest generated by the generation unit 32 on the display 24 in association with the second image of interest. On the screen D1, the comment on findings L21 regarding the nodule A21 (second region of interest) is displayed below the second image of interest T21.

The control unit 36 may also perform control to display comparison information between the first region of interest and the second region of interest generated by the generation unit 32 on the display 24. The comment on findings L21 on the screen D1 includes a character string indicating a variation in the size of the nodule (“It has increased compared to the previous time”), and an underline 95 is added thereto. In this way, in a case in which the control unit 36 displays a character string indicating comparison information on the display 24, the control unit 36 may highlight the character string, for example, by underlining it, changing the font, bolding, italics, or text color, etc.

Furthermore, the control unit 36 may receive additions and corrections by the user to the comment on findings including the finding information of the second region of interest generated by the generation unit 32. Specifically, the control unit 36 may perform control to display an input field for receiving a character string such as a comment on findings, including a description regarding the second image of interest, on the display 24 in association with the second image of interest. For example, in a case in which a “correct” button 97 or the icon 96 is selected by operating a mouse pointer 92 on the screen D1, the control unit 36 may display an input field for receiving the addition and correction of the comments on findings L21 in a display region 93 of the comments on findings L21 (not shown).

In addition, in a case in which the specifying unit 34 specifies a plurality of first regions of interest from a character string such as a comment on findings, the control unit 36 may perform control to display the first image of interest and the second image of interest specified for each of the plurality of first regions of interest on the display 24 in sequence. For example, in a case in which a “next” button 98 is selected on the screen D1, the control unit 36 may transition to a screen D2 displaying a first image of interest and a second image of interest specified for a first region of interest other than the nodule A11.

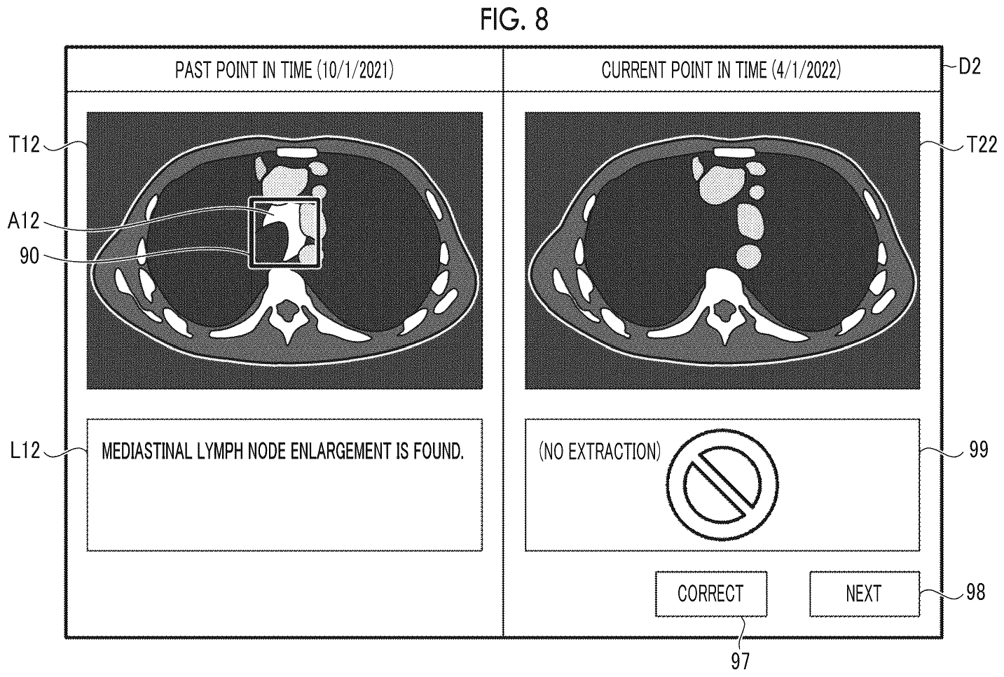

FIG. 8 shows an example of a screen D2 displayed on the display 24 by the control unit 36. On the screen D2, a first image of interest T12 including a mediastinal lymph node enlargement A12 (an example of a first region of interest) specified from the comment on findings L12 of FIG. 6, and a second image of interest T22 corresponding to the first image of interest T12 are displayed. On the screen D2, similarly to the screen D1, the mediastinal lymph node enlargement A12 in the first image of interest T12 is surrounded by a bounding box 90, and the comment on findings L12 regarding the mediastinal lymph node enlargement A12 is displayed below the first image of interest T12.

However, there are cases in which a second region of interest corresponding to the first region of interest included in the first image of interest is not necessarily included in the second image of interest. For example, if a lesion included in a first image of interest captured at a past point in time has healed by the current point in time, a second region of interest is not extracted from a second image of interest captured at the current point in time.

Therefore, in a case in which the second region of interest is not included in the second image of interest, that is, in a case in which the generation unit 32 is unable to extract the second region of interest from the second image of interest, the control unit 36 may provide a notification indicating that the second region of interest is not included in the second image of interest. As an example, on the screen D2, a notification 99 indicates that the second region of interest corresponding to the mediastinal lymph node enlargement A12 in the first image of interest T12 has not been extracted from the second image of interest T22. In this case, the generation unit 32 may omit generating a comment on findings regarding the second region of interest that could not be extracted. In this case, the control unit 36 may also omit displaying the second image of interest T22.

In addition, similarly to the screen D1, in a case in which the “correct” button 97 is selected on the screen D2, the control unit 36 may receive an input by the user for the comments on findings regarding the second image of interest T22. Also, similarly to the screen D1, in a case in which the “next” button 98 is selected on the screen D2, the control unit 36 may transition to a screen displaying a first image of interest and a second image of interest specified for a first region of interest other than the nodule A11 and the mediastinal lymph node enlargement A12. For example, the control unit 36 may perform control to display, on the display 24, a screen including a first image of interest including a hemangioma of the liver (an example of a first region of interest) specified from the comment on findings L13 in FIG. 6, and a second image of interest corresponding to the first image of interest (not shown).

In a case in which the first image of interest and the second image of interest are displayed in sequence as described above, the control unit 36 may perform control to display the first image of interest and the second image of interest on the display 24 in sequence in an order according to a predetermined priority for each of the plurality of first regions of interest.

The priority may be determined based on the position of the first image of interest, for example. For example, the priority may be lower from a head side to a waist side (that is, the priority may be higher closer to the head side). Furthermore, for example, the priority may be determined according to a guideline, a manual, or the like that specifies the order in which structures and/or lesions included in a medical image are to be interpreted.

Furthermore, for example, the priority may be determined according to at least one of the findings of the first region of interest or the second region of interest diagnosed based on at least one of the first image of interest or the second image of interest. For example, the worse the medical condition estimated based on at least one of the finding information of the first region of interest acquired by the acquisition unit 30 or the finding information of the second region of interest generated by the generation unit 32, the higher the priority may be.

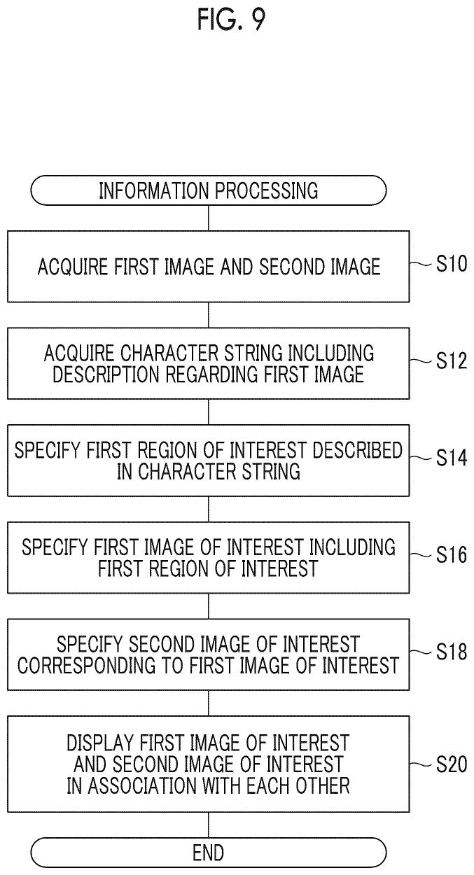

Next, with reference to FIG. 9, operations of the information processing apparatus 10 according to the present embodiment will be described. In the information processing apparatus 10, as the CPU 21 executes the information processing program 27, information processing shown in FIG. 9 is executed. The information processing is executed, for example, in a case in which the user provides an instruction to start execution via the input unit 25.

In Step S10, the acquisition unit 30 acquires at least one medical image (first image) obtained by imaging the subject at a past point in time, and at least one medical image (second image) obtained by imaging the subject at a current point in time. In Step S12, the acquisition unit 30 acquires a character string including a description regarding the first image acquired in Step S10.

In Step S14, the specifying unit 34 specifies a first region of interest described in the character string acquired in Step S12. In Step S16, the specifying unit 34 specifies a first image of interest including the first region of interest specified in Step S14 from the first image acquired in Step S10. In Step S18, the specifying unit 34 specifies a second image of interest corresponding to the first image of interest specified in Step S16 from the second image acquired in Step S10.

In Step S20, the control unit 36 performs control to display the first image of interest specified in Step S16 and the second image of interest specified in Step S18 on the display 24 in association with each other, and then ends this information processing.

As described above, the information processing apparatus 10 according to one aspect of the present disclosure comprises at least one processor. The processor acquires a character string including a description regarding at least one first image obtained by imaging a subject at a first point in time, specifies a first region of interest described in the character string, specifies a first image of interest including the first region of interest from the first image, specifies a second image of interest corresponding to the first image of interest from at least one second image obtained by imaging the subject at a second point in time, and displays the first image of interest and the second image of interest on a display in association with each other.

That is, with the information processing apparatus 10 according to the present embodiment, it is possible to perform comparative interpretation of a medical image (first image of interest) at a past point in time and a medical image (second image of interest) at a current point in time for a first region of interest described in an interpretation report at the past point in time. Therefore, it is possible to support the creation of an interpretation report at a current point in time.

In the above embodiment, a form has been described in which there is one first image of interest that is specified as including the first region of interest, and in a case in which there are a plurality of first regions of interest, the first images of interest including each of them are different from each other, but the present disclosure is not limited thereto. For example, the specifying unit 34 may specify a plurality of first images of interest including a certain first region of interest (for example, a nodule in a lung field). Also, for example, the specifying unit 34 may specify the same image as the first image of interest including each of a plurality of first regions of interest (for example, a nodule in a lung field and a mediastinal lymph node enlargement).

In addition, in the above embodiment, a form has been described in which the generation unit 32 performs image analysis on the second image to generate finding information of the second region of interest and to generate a character string such as a comment on findings including the finding information, but the present disclosure is not limited thereto. For example, the generation unit 32 may acquire finding information stored in advance in the storage unit 22, the image server 5, the image DB 6, the report server 7, the report DB 8, and other external devices. Alternatively, for example, the generation unit 32 may acquire finding information manually input by the user via the input unit 25.

Furthermore, for example, the generation unit 32 may acquire a character string such as a comment on findings stored in advance in the storage unit 22, the report server 7, the report DB 8, and other external devices. Furthermore, for example, the generation unit 32 may receive a character string such as a comment on findings manually input by the user. For example, the generation unit 32 may generate a plurality of candidates for character strings, such as comments on findings including finding information of the second region of interest, and allow the user to select which of the plurality of candidates to employ.

In addition, in the above embodiment, a form has been described in which a first image of interest and a second image of interest are specified and displayed for all first regions of interest included in a character string such as a comment on findings acquired by the acquisition unit 30, but the present disclosure is not limited thereto. For example, the control unit 36 may receive a selection of a portion of the character string such as a comment on findings acquired by the acquisition unit 30 to be used by the specifying unit 34 to specify the first region of interest.

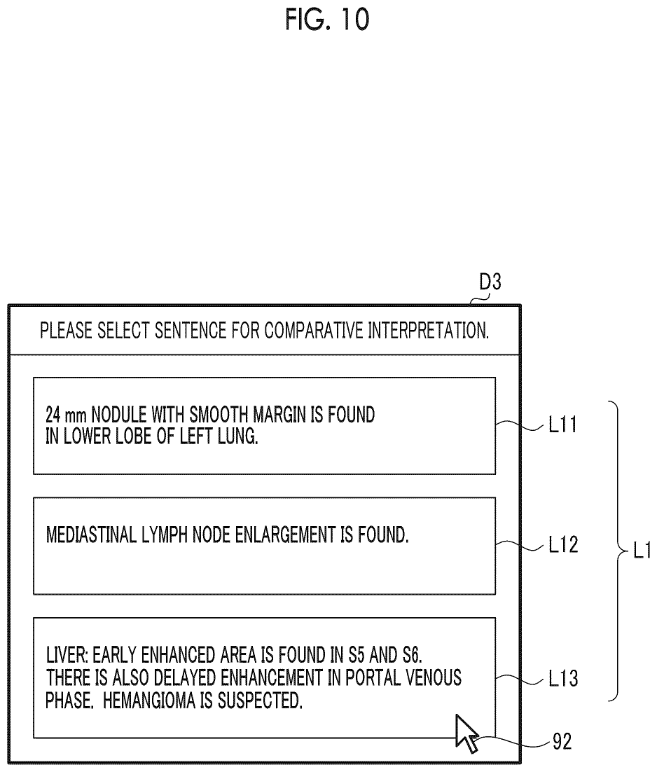

As an example, FIG. 10 shows a screen D3 for selecting a portion of the comment on findings L1. As shown on the screen D3, the control unit 36 may display the comments on findings L11 to L13 obtained by dividing the comment on findings L1 into regions of interest (lesions, structures, etc.) on the display 24, and receive the selection of at least one of the comments on finding L11, L12, or L13. The user operates the mouse pointer 92 to select at least one of the comments on finding L11, L12, or L13 displayed on the screen D3.

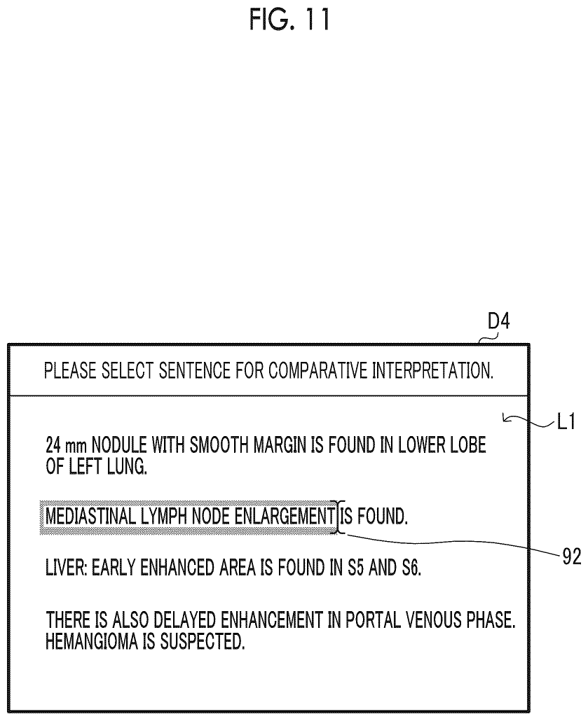

As another example, FIG. 11 shows a screen D4 for selecting a portion of the comment on findings L1. As shown on the screen D4, the control unit 36 may display the comment on findings L1 on the display 24 and receive the selection of any portion of the comment on findings L1. The user operates the mouse pointer 92 to select any portion of the comment on findings L1 displayed on the screen D4.

In the above embodiment, a form has been described in which the first image of interest and the second image of interest specified for each of the plurality of first regions of interest are displayed in sequence, but the present disclosure is not limited thereto. For example, the control unit 36 may perform control to display the first image of interest and the second image of interest specified for each of the plurality of first regions of interest on the display 24 as a list.

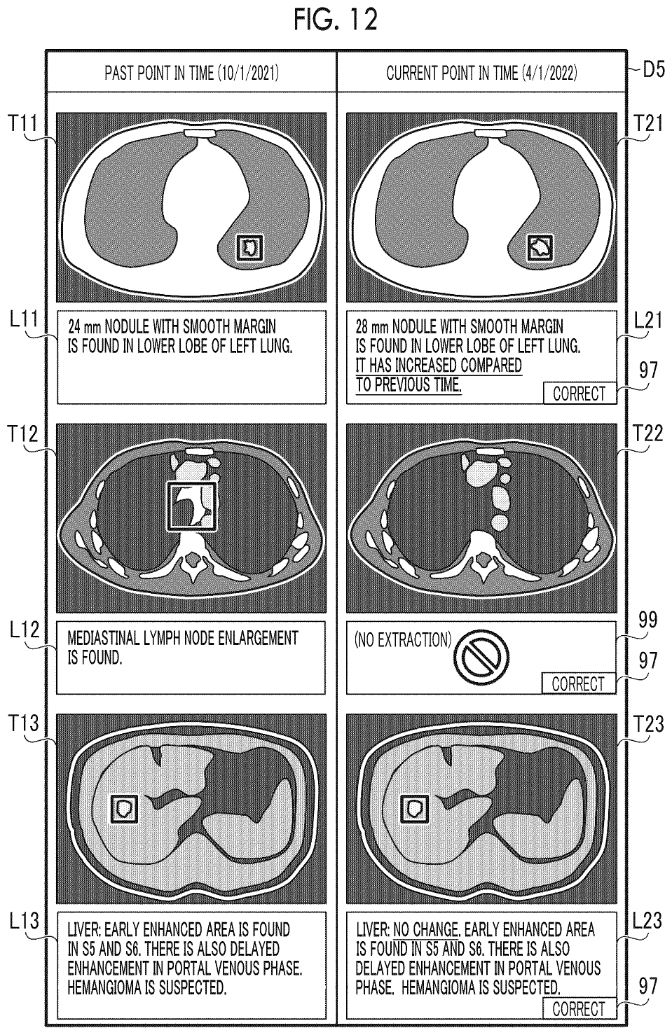

As an example, FIG. 12 shows a screen D5 in which the first images of interest T11 to T13 and the second images of interest T21 to T23 specified for each of the plurality of first regions of interest are displayed in a list format. As shown on the screen D5, the control unit 36 may perform control to display a plurality of comments on findings L11 to L13 on the display 24 in association with the plurality of first images of interest T11 to T13, respectively. In addition, the control unit 36 may perform control to display, on the display 24 in association with the plurality of second images of interest T21 to T23, the comments on findings L21 and L23 and a notification 99 indicating that the second region of interest is not included.

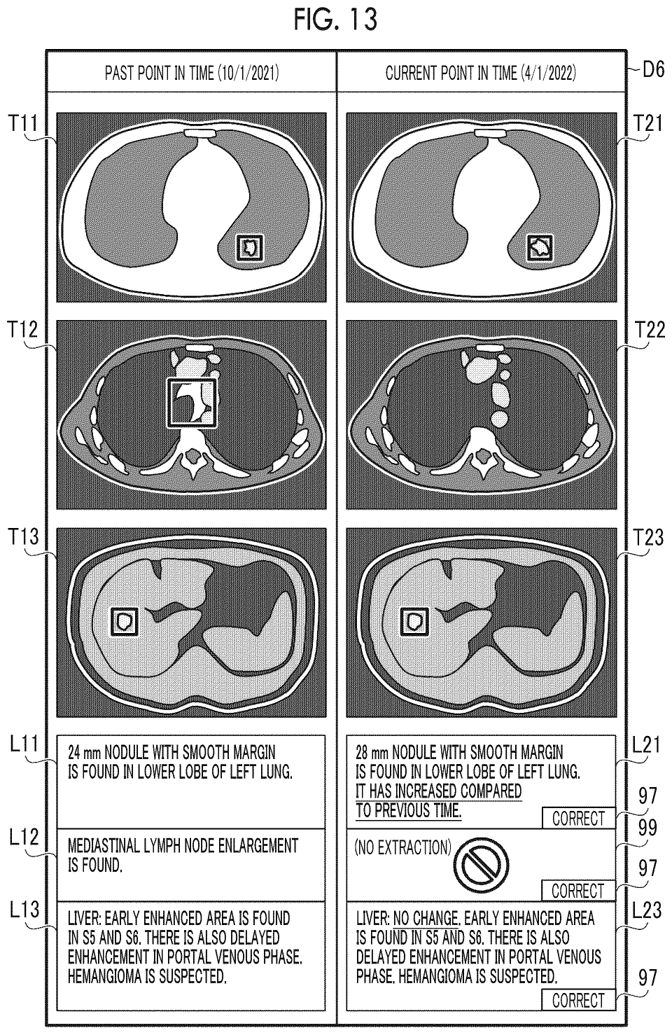

In addition, FIG. 13 shows a screen D6 as a modification example of the screen D5. On the screen D6, the first images of interest T11 to T13 and the second images of interest T21 to T23 are collectively shown at an upper part, and the comments on findings L11 to L13, L21, and L23 and a notification 99 indicating that the second region of interest is not included are collectively shown at a lower part.

In addition, in a case in which the control unit 36 displays the first image of interest and the second image of interest as a list, the control unit 36 may list the first image of interest and the second image of interest in an order according to a predetermined priority for each of the plurality of first regions of interest. For example, the control unit 36 may rearrange the first image of interest and the second image of interest such that the upper part of the screen D5 is on the head side and the lower part thereof is on the waist side. Furthermore, for example, the control unit 36 may rearrange the first image of interest and the second image of interest in an order in which the medical conditions of the first region of interest and/or the second region of interest are estimated to be worse.

In addition, in the embodiment described above, a form has been described in which a first image of interest and a second image of interest specified for a first region of interest other than the first region of interest being displayed are displayed in a case in which the “next” button 98 is selected on the screen D1 and the screen D2, but the present disclosure is not limited thereto. For example, after receiving a character string (comment on findings L21) such as a comment on findings including a description regarding the second image of interest in an input field displayed on the screen D1, the control unit 36 may perform control to display the first image of interest and the second image of interest specified for a next first region of interest on the display 24. In other words, at the point in time at which the addition and correction of the comment on findings L21 are completed, the screen may automatically transition to the screen D2 displaying a first image of interest and a second image of interest specified for a first region of interest other than the nodule A11.

In the above embodiment, a lesion that was not included in the first image may be included in the second image. For example, there may be cases in which a lesion that did not exist at a past point in time has newly appeared by the current point in time and can be extracted from a second image captured at the current point in time. Therefore, the specifying unit 34 may specify the region of interest that was not included in the first image by performing image analysis on the second image. In addition, the control unit 36 may provide a notification indicating that a region of interest that was not included in the first image has been detected from the second image.

Further, in the above embodiment, a form assuming an interpretation report for medical images has been described, but the present disclosure is not limited thereto. The information processing apparatus 10 according to one aspect of the present disclosure is applicable to various documents including descriptions regarding images obtained by imaging a subject. For example, the information processing apparatus 10 may be applied to documents including descriptions regarding images acquired using an apparatus, a building, a pipe, a welded portion, or the like as a subject in a non-destructive examination such as a radiation transmission examination and an ultrasonic flaw detection examination.

In addition, in the above embodiment, for example, as hardware structures of processing units that execute various kinds of processing, such as the acquisition unit 30, the generation unit 32, the specifying unit 34, and the control unit 36, various processors shown below can be used. As described above, the various processors include a programmable logic device (PLD) as a processor of which the circuit configuration can be changed after manufacture, such as a field-programmable gate array (FPGA), a dedicated electrical circuit as a processor having a dedicated circuit configuration for executing specific processing such as an application-specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system-on-chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiment, the information processing program 27 is described as being stored (installed) in the storage unit 22 in advance; however, the present disclosure is not limited thereto. The information processing program 27 may be provided in a form recorded in a recording medium such as a compact disc read-only memory (CD-ROM), a digital versatile disc read-only memory (DVD-ROM), and a Universal Serial Bus (USB) memory. In addition, the information processing program 27 may be configured to be downloaded from an external device via a network. Further, the technology of the present disclosure extends to a storage medium for storing the information processing program non-transitorily in addition to the information processing program.

The technology of the present disclosure can be appropriately combined with the above embodiment and examples. The described contents and illustrated contents shown above are detailed descriptions of the parts related to the technology of the present disclosure, and are merely an example of the technology of the present disclosure. For example, the above description of the configuration, function, operation, and effect is an example of the configuration, function, operation, and effect of the parts related to the technology of the present disclosure. Therefore, it goes without saying that unnecessary parts may be deleted, new elements may be added, or replacements may be made to the described contents and illustrated contents shown above within a range that does not deviate from the gist of the technology of the present disclosure.

The disclosure of JP2022-065907 filed on Apr. 12, 2022 is incorporated herein by reference in its entirety. All documents, patent applications, and technical standards described in the present specification are incorporated in the present specification by reference to the same extent as in a case in which each of the documents, patent applications, and technical standards are specifically and individually indicated to be incorporated by reference.

What is claimed is:

1. An information processing apparatus comprising a processor, wherein the processor is configured to:
acquire a character string including a description regarding at least one first image obtained by imaging a subject at a first point in time;
specify a first region of interest described in the character string;
specify a first image of interest including the first region of interest from the first image;
specify a second image of interest corresponding to the first image of interest from at least one second image obtained by imaging the subject at a second point in time; and
display the first image of interest and the second image of interest on a display in association with each other.

2. The information processing apparatus according to claim 1, wherein the processor is configured to specify the second image obtained by imaging the same position as the first image of interest as the second image of interest.

3. The information processing apparatus according to claim 1, wherein the processor is configured to receive a selection of a portion of the character string to be used to specify the first region of interest.

4. The information processing apparatus according to claim 1, wherein the processor is configured to, in a case in which a plurality of the first regions of interest described in the character string are specified, specify the first image of interest and the second image of interest for each of the plurality of first regions of interest.

5. The information processing apparatus according to claim 4, wherein the processor is configured to display the first image of interest and the second image of interest specified for each of the plurality of first regions of interest on the display in sequence.

6. The information processing apparatus according to claim 5, wherein the processor is configured to display the first image of interest and the second image of interest on the display in sequence in an order according to a predetermined priority for each of the plurality of first regions of interest.

7. The information processing apparatus according to claim 5, wherein the processor is configured to:
display an input field for receiving a character string including a description regarding the second image of interest on the display in association with the second image of interest; and
display a next first image of interest and a next second image of interest on the display after receiving the character string including the description regarding the second image of interest in the input field.

8. The information processing apparatus according to claim 4, wherein the processor is configured to display the first image of interest and the second image of interest specified for each of the plurality of first regions of interest on the display as a list.

9. The information processing apparatus according to claim 1, wherein the processor is configured to notify a user to check a region corresponding to the first region of interest in the second image of interest.

10. The information processing apparatus according to claim 9, wherein the processor is configured to display a character string indicating the first region of interest and at least one of a symbol or a figure on the display as the notification.

11. The information processing apparatus according to claim 1, wherein the processor is configured to:
generate comparison information indicating a result of comparing the first region of interest in the first image of interest with a region corresponding to the first region of interest in the second image of interest; and
display the comparison information on the display.

12. The information processing apparatus according to claim 1, wherein the processor is configured to highlight a region corresponding to the first region of interest in the second image of interest.

13. The information processing apparatus according to claim 1, wherein the processor is configured to display a character string including a description regarding at least the first region of interest on the display in association with the first image of interest.

14. The information processing apparatus according to claim 1, wherein the processor is configured to display an input field for receiving a character string including a description regarding the second image of interest on the display in association with the second image of interest.

15. The information processing apparatus according to claim 1, wherein the processor is configured to display the first image of interest and the second image of interest on the display with the same display settings.

16. The information processing apparatus according to claim 15, wherein the display settings are settings related to at least one of a resolution, a gradation, a brightness, a contrast, a window level, a window width, or a color of the first image of interest and the second image of interest.

17. The information processing apparatus according to claim 1, wherein the processor is configured to, in a case in which a region corresponding to the first region of interest is not included in the second image of interest, provide a notification indicating that the region corresponding to the first region of interest is not included in the second image of interest.

18. The information processing apparatus according to claim 1, wherein
the first image and the second image are medical images, and
the first region of interest is at least one of a region of a structure included in the medical image or a region of an abnormal shadow included in the medical image.

19. An information processing method comprising:
acquiring a character string including a description regarding at least one first image obtained by imaging a subject at a first point in time;
specifying a first region of interest described in the character string;
specifying a first image of interest including the first region of interest from the first image;
specifying a second image of interest corresponding to the first image of interest from at least one second image obtained by imaging the subject at a second point in time; and
displaying the first image of interest and the second image of interest on a display in association with each other.

20. A non-transitory computer-readable storage medium storing an information processing program for causing a computer to execute a process comprising:
acquiring a character string including a description regarding at least one first image obtained by imaging a subject at a first point in time;
specifying a first region of interest described in the character string;
specifying a first image of interest including the first region of interest from the first image;
specifying a second image of interest corresponding to the first image of interest from at least one second image obtained by imaging the subject at a second point in time; and
displaying the first image of interest and the second image of interest on a display in association with each other.
